(19) United States
(12) Patent Application Publication
(10) Pub. No.: US 2014/0371617 A1
(43) Pub. Date: Dec. 18, 2014

(54) REMOTE HEALTH CARE DIAGNOSTIC TOOL

(71) Applicant: Rajiv Muradia, Ottawa (CA)

(72) Inventor: Rajiv Muradia, Ottawa (CA)

(21) Appl. No.: 14/180,112

(22) Filed: Feb. 13, 2014

Related U.S. Application Data
(63) Continuation of application No. 13/400,473, filed on Feb. 20, 2012, now abandoned.
Application No. 13/400,473 is a continuation of application No. 11/756,956, filed on Jun. 1, 2007, now abandoned.
(60) Provisional application No. 60/809,839, filed on Jun. 1, 2006.

(51) Int. Cl. G06F 19/00; A61B 5/024; A61B 7/02; H04L 29/08

(52) U.S. Cl. CPC G06F 19/3418; G06F 19/322; H04L 67/142; A61B 5/024; A61B 7/02
USPC 600/528; 705/3

(57) ABSTRACT

Disclosed are patient health systems and methods for transmission of patient health data from a patient data collection system to a provider analysis system. The patient health system include the patient data collection system coupled to a communications network and located proximate a patient, for collecting patient physiological data. The patient health system further includes the provider analysis system coupled to the communications network and located remote from the patient data collection system. The patient data collection system includes a patient data collection device couple to a patient work station. The patient work station is configured to transmit patient physiological data upon a determination that the communications network is reliable.

### CROSS-REFERENCE TO RELATED APPLICATIONS

This application is a continuation of application Ser. No. 13/400,473, filed Feb. 20, 2012, which is a continuation of application Ser. No. 11/756,956, filed Jun. 1, 2007, which claims the benefit of provisional application Ser. No. 60/809,839, filed Jun. 1, 2006, which applications are incorporated herein by reference in their entirety.

### FIELD OF THE INVENTION

The invention relates to remote health care systems and more specifically, to remote monitoring of patient environment with environmental sensors that transmit environmental data to a central server over a communications network.

### BACKGROUND

In recent years, the costs of providing high quality health care have increased to the point that, in many countries, health care costs represent a significant portion of state expenditures. In some jurisdictions private health care companies provide health care services. In both cases increasing costs of skilled medical professionals, medical test equipment and pharmaceuticals have resulted in strong desire to find inexpensive alternatives.

One way to provide improved health care without the cost of keeping patients in a hospital is to have patients return home and complete their health care program at home. Such systems take advantage of inexpensive medical testing sensors such as those described by Boecker et al. in U.S. Pat. No. 6,966,880. In some cases it remains necessary to monitor specific medical criteria of patients and the prior art teaches providing medical test equipment in the home of a patient, monitoring the patient with the medical test equipment to generate medical data, providing the medical data to a medical service and monitoring the condition of the patient. Such a system is taught by Ridgeway in U.S. Pat. No. 5,976,975.

The prior art teaches a variety of useful home care techniques however as is well understood in the art, the cost of dealing with many medical conditions increases with the length of time that the condition goes undiagnosed. In addition, the rate of success in treating a variety of diseases improves with early detection.

Unfortunately, it is often the case that people who are suffering medically treat their health as a relatively low priority. This lack of desire to deal with one's health often allows a medical condition to progress from a minor annoyance to a disease that disrupts the life of the patient and, as a result of its progression, is more difficult to treat. Conversely, some people are inclined to visit a doctor at the slightest sign of a medical issue. In many cases such people allow their own worries about medical problems to affect their judgment regarding the severity of any disruption of their own wellness.

It would be beneficial to provide an enhanced remote medical system that assists medical professionals in monitoring the health of a patient absent having the patient consult with a medical professional directly.

### SUMMARY

It is to be understood that both the foregoing general description and the following detailed description are exemplary and explanatory only, and should not be considered restrictive of the scope of the invention, as described and claimed. Further, features and/or variations may be provided in addition to those set forth herein. For example, embodiments of the invention may be directed to various combinations and sub-combinations of the features described in the detailed description and include systems and methods for transmission of patient heart beat data from a patient work station to a remote health provider analysis system.

### BRIEF DESCRIPTION OF THE DRAWINGS

The invention is now described with reference to the drawings in which:

FIG. 1 is a diagram of an embodiment of the invention;

FIG. 2A. is an illustration of the process and flow of data that occurs during patient use of the system illustrated in FIG. 1;

FIG. 2B. is further illustration of the process and flow of data that occurs during patient use of the system illustrated in FIG. 1;

FIG. 3. is an illustration of the process and flow of data that occurs during care provider use of the system illustrated in FIG. 1;

FIG. 4. illustrates a login screen;

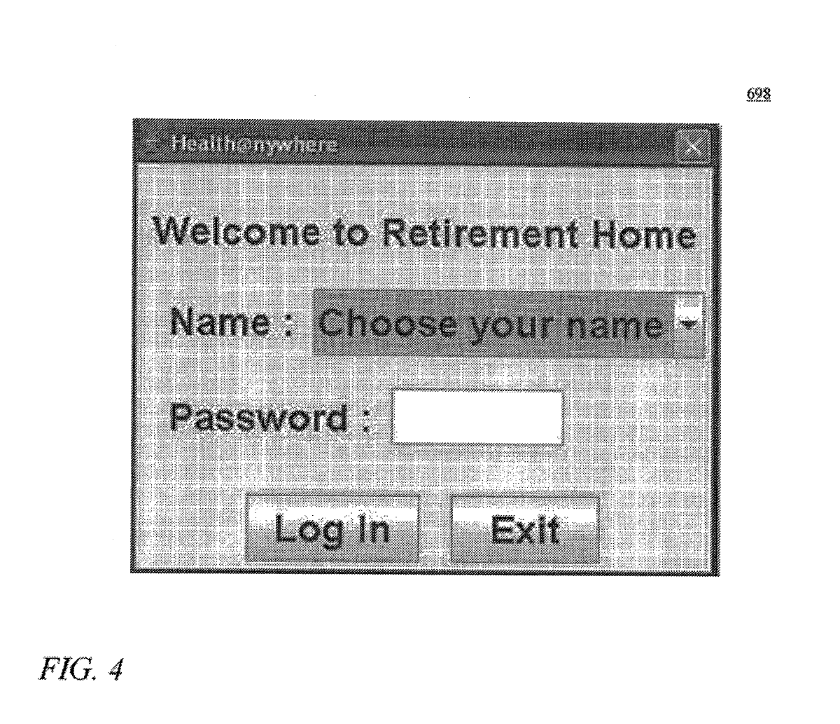

FIG. 5. illustrates an access welcome screen;

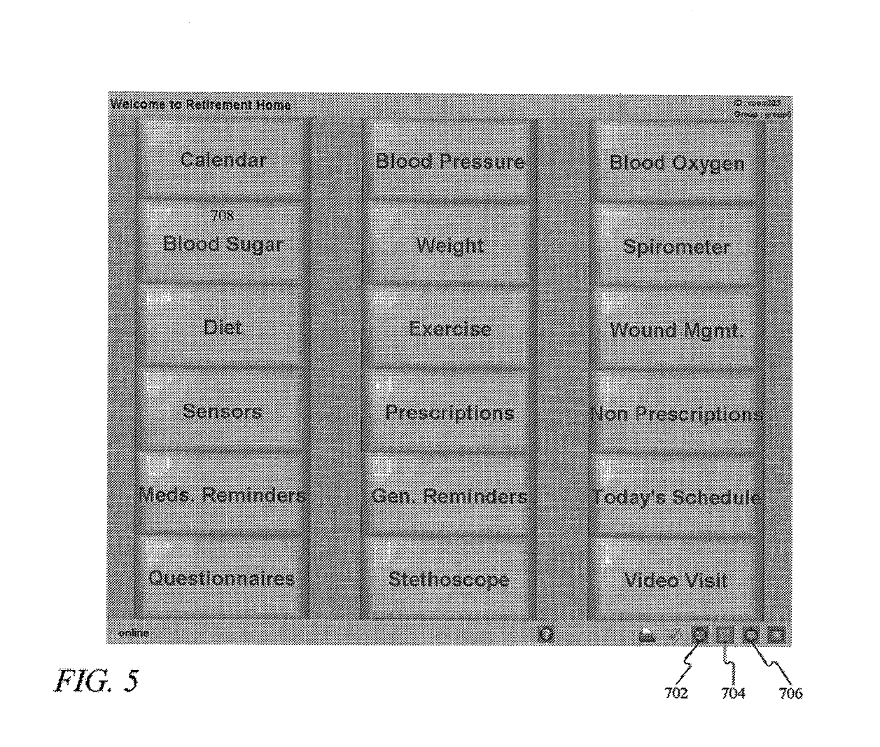

FIG. 6. illustrates a blood sugar monitoring screen;

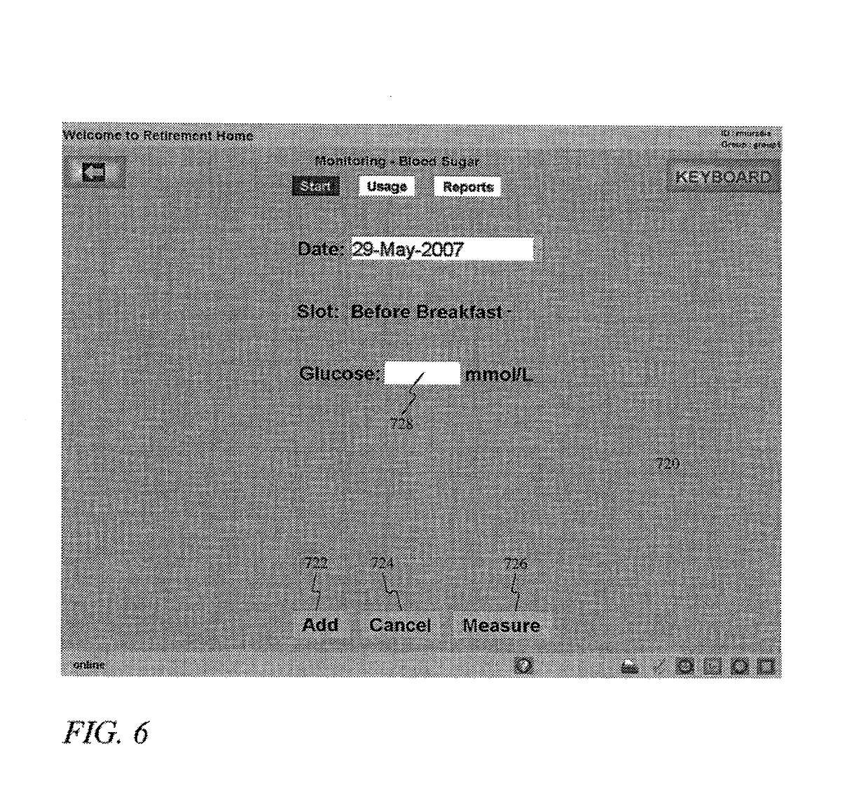

FIG. 7. illustrates a blood sugar monitoring screen;

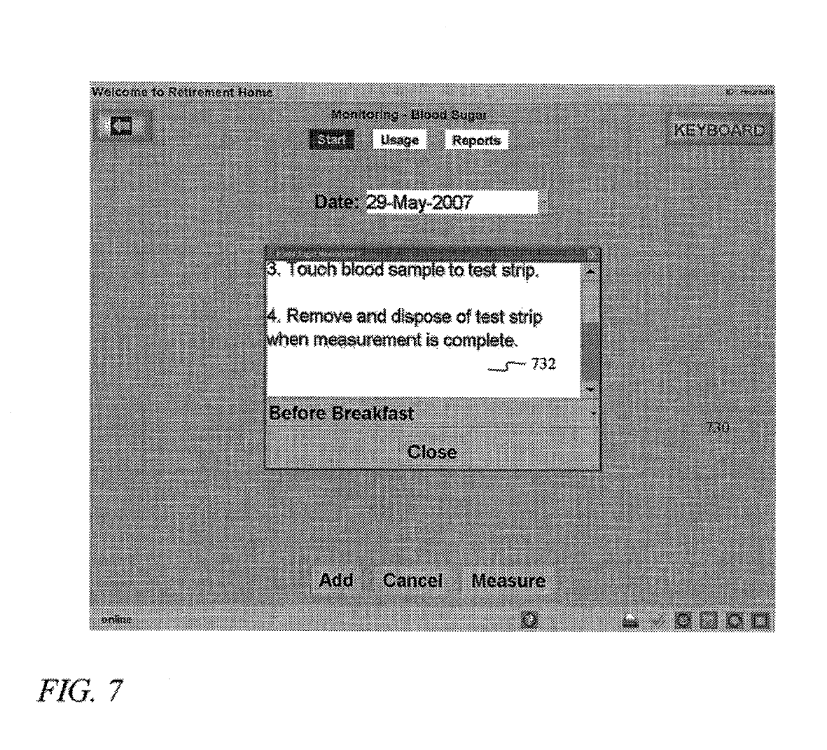

FIG. 8. illustrates an instruction screen;

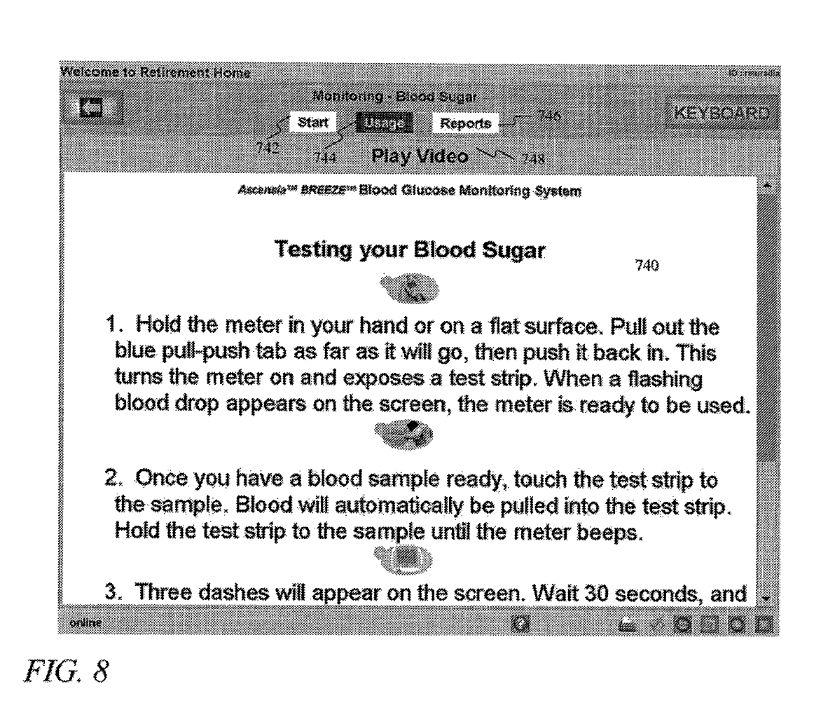

FIG. 9. illustrates a video box;

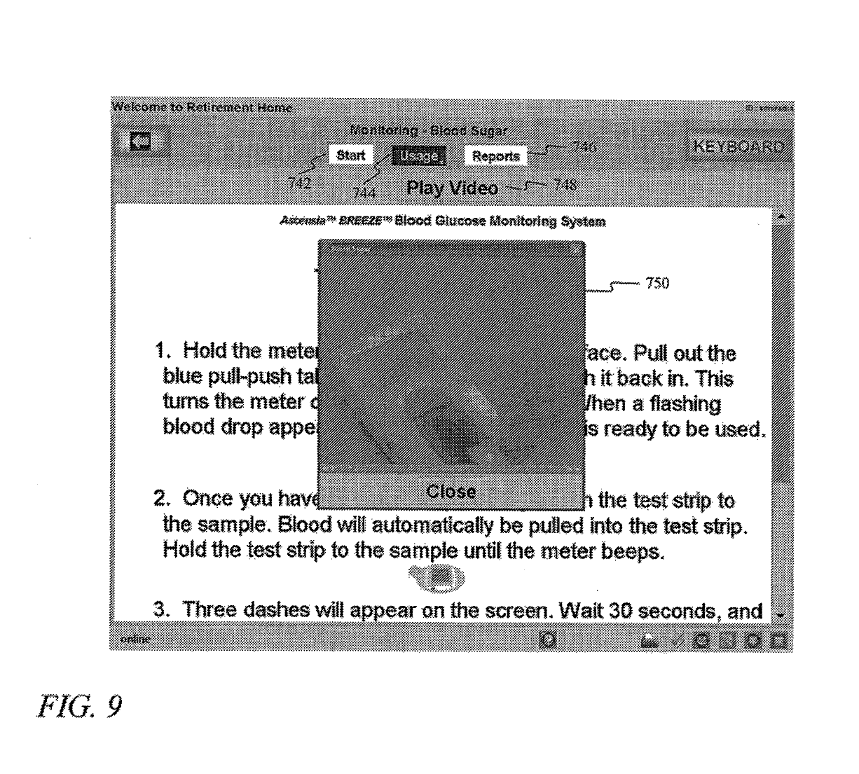

FIG. 10. illustrates a report screen; and

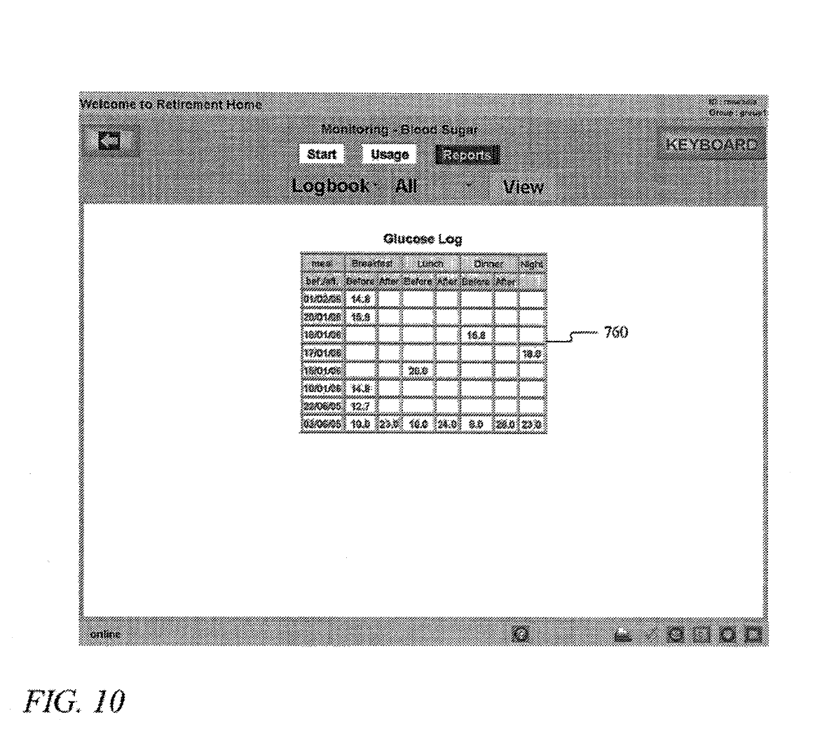

FIG. 11. illustrates a graph of measurements.

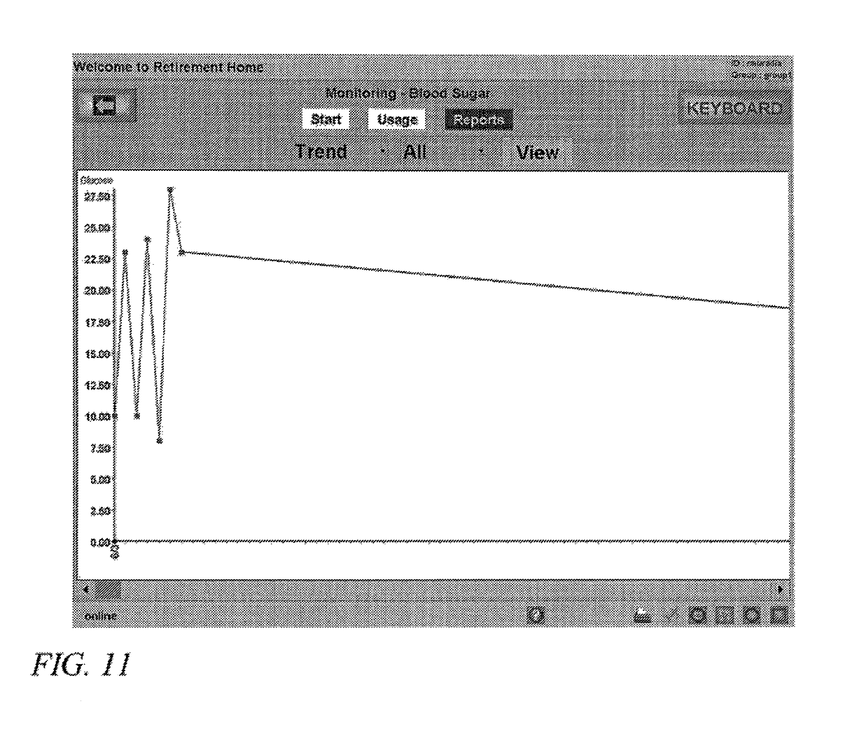

### DETAILED DESCRIPTION OF THE INVENTION

The following detailed description refers to the accompanying drawings. Wherever possible, the same reference numbers are used in the drawings and the following description to refer to the same or similar parts. While several exemplary embodiments and features of the invention are described herein, modifications, adaptations and other implementations are possible, without departing from the spirit and scope of the invention. For example, substitutions, additions or modifications may be made to the components illustrated in the drawings, and the exemplary methods described herein may be modified by substituting, reordering or adding steps to the disclosed methods. Accordingly, the following detailed description does not limit the invention. Instead, the proper scope of the invention is defined by the appended claims.

The present invention relates to systems and methods in the remote health care environment. Systems and methods consistent with embodiments of the present invention may be used to transmit data representative of a patient's environment from a patient health station to which a plurality of environmental sensors are operatively connected in order to facilitate automatic transmission of patient environment data. The system and method may be facilitated by providing environmental sensors proximate the patient to sense information about the patient such as air quality, location within the home etc. Environmental sensors are passive, meaning they do not require a patient's engagement in order to facilitate retrieval and loading of environmental data onto the patient health station. The patient work station processes the data representative of a patient's environment to determine if the environmental data is within predetermined acceptable ranges. Data representative of a patient's environment that is not within acceptable ranges is flagged with an indicator and stored in a memory buffer. The data is flagged with an indicator so that a health care provider can easily pinpoint such data during assessment. Upon a determination by the patient health station that the communications medium between the patient health station and the health care provider analysis system is sufficiently stable, the data within the memory buffer is transmitted in gradual and orderly fashion. Gradual and orderly data transmission helps to facilitate recovery and redistribution of incomplete data transmissions resulting from communication network service interruptions.

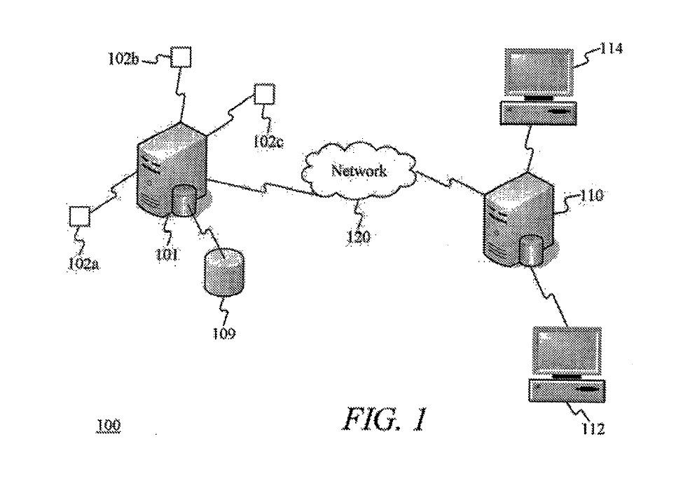

Consistent with an embodiment of the present invention, the aforementioned patient health station and health care provider analysis system which are operatively coupled via a communications network may be implemented in the embodiments illustrated in FIG. 1. Referring to FIG. 1, a system according to the first embodiment of the invention is shown. The system 100 supports communication between a patient health station 101 and a central server 110 of a health care provider or data hosting organization that is remote to the patient health station 101 and operatively connected via communications medium 120. The patient health station 101 is operatively connected to a set of sensors 102a, 102b, 102c disposed in a patients living quarters for the purpose of monitoring the environment of the living quarters. The set of sensors communicate with the patient health station 101 which in turn communicates with the central server that is accessible by a medical professional. In use, following installation and synchronization of the set of sensors 102a, 102b, 102c with the patient health station 101, the sensors are activated and facilitate transmission of patient environmental data to the central server 110. When the patient environmental data is indicative of the worsening of a healthcare condition and or the initiation of previously unknown medical conditions, the medical professional whom has access to the patient health data may be notified and in response may contact the patient.

The set of sensors 102a, 102b, 102c collect data absent interaction with the patient. More specifically, the sensors of the set of sensors 102a, 102b, 102c provide data regarding the health of the patient absent the patient or others providing direct medical inputs to the system. Thus, for example, a first sensor disposed in the patient's bed monitors the duration and quality of the patient's sleep. A second sensor monitors use of toilets in the home of the patient. Optionally, the toilet comprises a sensor that identifies the patient from a plurality of other users of the toilet. In addition, such a sensor optionally provides chemical analysis data associated with the contents of the toilet. Further optionally, another sensor monitors the opening of doors to the outside of the living quarters. In this way, if a patient who is known to suffer from depression is known to have been in bed for extended periods of time but has not gone out of their home a medical professional is alerted to the situation. Similarly, the operation of appliances within the household of the patient is similarly monitored.

Clearly, the first embodiment of the invention supports the use of a wide variety of sensors that passively monitor certain actions of the patient. Some passive sensors which may be used in the present invention include sensors that monitor sleep, bathroom visits, bedroom visits, activity monitoring, meal preparation, air quality and patient fall status. A sensor that monitors a patient sleep may be comprised of a pad to detect breathing pattern, or a pad that detects movement during sleep or whether a patient is getting in/out of bed. Such a sensor may be connected via wireless or wired connection. A sensor that monitors bathroom visits may be door contacts, toilet seat contacts. A sensor that monitors bedroom visits may be wired or wireless door contacts. A sensor that monitors the patient's activity level may be sensors throughout the house, such as wired or wireless contacts on doors. A sensor that monitors the patient's meal preparation may be sensors that detect the opening and closing of stove, refrigerator, microwave oven, etc. A sensor that monitors the quality of air in a patient's home may be comprised of a sensor that can monitor a number of different components such as CO2 levels, pollen count etc. A sensor that monitors a patient's fall status detects the position of the body relative to ground. Any of the sensors utilized to monitor patient environment may be connected by hard wire or via wirelessly.

A person of skill in the art will be aware of other sensors that collect data in a passive way that are also useful in assessing the health of an individual. In addition, it is beneficial to acquire data relating to the environment in which the patient lives absent independent of collecting medical information about the patient for the purpose of determining a patient's health. For example, an environmental sensor disposed within a patient's home monitors air quality. If the patient is known to have a breathing disorder such as asthma, then it is valuable to have data relating to the quality of the air that the patient is breathing. Thus, if the patient reports that they are experiencing more difficulty breathing than would be the case usually, that medical professional is able to determine if the problem is likely associated with a recent change in air quality.

A person of skill in the art will also appreciate that the use of specific sensors involves a certain degree of user interaction. For example, a sensor that monitors air quality is likely to support functions that monitor air quality absent input signals from the patient once the sensor is suitably located and configured. In contrast, a scale that the patient stands on to activate requires activation by the patient. Thus, while a scale provides very useful medical information, it is not truly a passive sensor because the patient interacts with the scale in order to provide a reading from the scale. In contrast, while a toilet requires some interaction by the patient, ie. both use and flushing, that interaction is understood to be common practice associated with normal bodily functions. Clearly, in most technological societies, there is no convenient sanitary alternative to using a toilet and therefore the interaction of the patient and the toilet is understood to be normal practice. In this way, a sensor configured to report the use of a toilet involves no unconventional interaction between the patient and the toilet. In contrast, a person who is not accustomed to standing on a scale may simply forget to do so.

Clearly, the first embodiment of the invention supports determining when a patient should seek skilled medical attention. In addition, the first embodiment of the invention allows an individual to have their health monitored automatically by a medical professional. The medical professional accesses data within the central server 110 via a workstation 112, 114. In an alternative to the first embodiment of the invention, the server 110 includes non-volatile memory. The non-volatile memory is used to store data in dependence upon information received from the sensors. In this way, the medical professional is able to review a history of data regarding the patient and thereby provide more accurate assessments of the patient's current health.

A person of skill in the art will appreciate that there are a variety of techniques for supporting data transmission from the sensors of the set of sensors 102a, 102b, 102c to the central server 110 are optionally supported. For example, in some cases it is convenient to provide sensors that support wireless data transmission, such as Bluetooth sensors, that may communicate with the patient station 101 which also supports receiving wireless data from the sensors. The patient station 101 having received the wireless data then transmits the data to the central server 110 via network connection 120, which may be for example, an Internet connection. Alternatively, the set of sensors 102a, 102b, 102c provide data to the patient station 101 that has the capability to send patient data over a cellular network. A variety of different communications infrastructure is optionally used as the communications medium 120. For example, the terminal 101 optionally communicates with the server 110 via a wireless link, an Internet link or a plain old telephone system (POTS).

Optionally, identification data is captured along with the sensor data in order to allow use of the system in a home occupied by more than one individual. For example, a toilet is fit with a weight measure for distinguishing between members of a household. Further optionally, the method is employed in buildings other than homes. For example, in an office building a urinal is fit with a sensor for sensing urine content and with a second sensor for sensing identifying information about the origin of the urine.

As FIG. 1 illustrates, the patient station 101 includes a memory buffer 109 disposed electronically proximate the patient station 101. The memory buffer 109 supports receiving data from the passive sensors 102. The memory buffer 109 is optionally located within the patient station 101 or external to it. In use, a passive sensor 102 automatically transmits environmental data to the patient station 101 which facilitates the storage of patient environmental data received in the memory buffer 109. A person of skill in the art will appreciate that the ability to support communications between remote locations is often difficult to achieve in practice, particularly in areas that are not well served. The memory buffer 109 serves to mitigate such problems by storing information associated with the environmental data received from sensors 102 and transmitting it to the central server 110 via network connection 120 when the communications network supports such data transfer.

When patient environmental data is being transmitted between the patient station 101 and the central server 110, the data is stored in the memory buffer 109 and transferred to the central server 110 in a gradual fashion that supports verification of the accuracy of the patient environmental data being provided. In this way, should communication between the patient station 101 and the central server 110 fail, the information regarding the patient environmental data is still available. Optionally, the memory buffer 109 supports downloading of data stored therein via a local communications port, such as a universal serial bus (USB) port. A person of skill in the art will appreciate that buffering and then transmitting the patient environmental data will require more time than simply sending the patient environmental data directly. Clearly, in situations that allow the patient environmental data to be transmitted directly it is still beneficial to temporarily and simultaneously store the patient environmental data in the memory buffer 109 as even robust communications links are subject to temporary reductions in bandwidth and other types of failure. Optionally, the data transmitted is transmitted in a compressed form.

Further optionally, the patient station 101 may include predetermined medical instructions regarding how a patient operates a passive sensor such as a stethoscope. When the patient accesses the patient station 101 they identify themselves. The computing device within the patient station 101 interprets data within the non-volatile memory and provides the predetermined medical instructions to the patient in accordance with the data. The patient then operates a passive sensor, such as an electronic stethoscope by recording their heartbeat in accordance with the instructions provided. Data within the memory buffer is later transmitted to the central server 110.

In addition, the patient station 101 supports additional active sensors such as medical testing equipment, which monitor such things as a heart rate monitor and blood glucose meter, to name a few. Such instruments are designed to support providing measured health information to the central server 110. A person of skill in the art will appreciate that this embodiment of the invention is easily modified to support a wide variety of medical tests.

The patient station 101 may also include a video screen for providing visual information. In use, the medical professional is able to provide video information to the patient. The information provided to the patient will most likely be in the context of an analysis of all patient medical data which includes data automatically transmitted by the passive sensors 102. Thus, should the patient experience some difficulty with a self-administered medical procedure that is performed through the use of active sensors 104, the medical professional is able to provide the patient relevant instruction both visually and audibly in order to assist the patient. Optionally, the medical professional provides a predetermined video stream to the patient station 101 where the media stream comprises medical instruction information for the purpose of instructing a patient regarding a self administered medical procedure. Further optionally, a set of such procedures are stored in a non-volatile storage memory proximate the server 110.

A person of skill in the art will appreciate that there are a wide variety of techniques for using an active sensor 104 such as a stethoscope. While one embodiment of the invention features a stethoscope that comprises a microphone that supports recording of heartbeat data to an external medium, an alternative stethoscope comprises an electronic microphone that is placed in close proximity to the patient's chest. In an alternative embodiment, the stethoscope comprises an elastic loop with a microphone that the patient positions against their skin proximate the heart with the elastic loop going around the chest. Such an embodiment optionally comprises a tension sensor for providing information regarding the amount of tension used to hold the sensor against the chest. The tension sensor facilitates the sensors ability to provide relatively consistent measurements. Further optionally, video transmission of the patient wearing the stethoscope is recorded and transmitted so that if the stethoscope is poorly located the medical professional will be able to easily verify this and redirect the patient concerning proper positioning.

It will be apparent to one of skill in the art that in many cases it is beneficial to have a medical professional other than a physician review patient environmental data and data captured from other active and passive sensors. Following a review of data, should the medical professional suspect that there is a health problem, they can transmit the relevant portions of data to a physician or other specialist for assessment. In this way, the medical professional and the cardiologist have the opportunity to review the suspect data retrieved from the environment along with data from active and passive sensors. This has the additional benefit of teaching the medical professional the characteristics of a suspect heartbeat.

A person of skill in the art will appreciate that a wide variety of techniques are available to support communication between the patient station 101 and the central server 110. Clearly, the choice of the technologies used is dependent upon a variety of factors, many of which are outside the scope of the present invention. Further, a person of skill in the art will appreciate that the embodiments of the invention presented are intended to be illustrative of the invention and not limiting. Numerous other embodiments of the invention will be apparent to one of skill in the art.

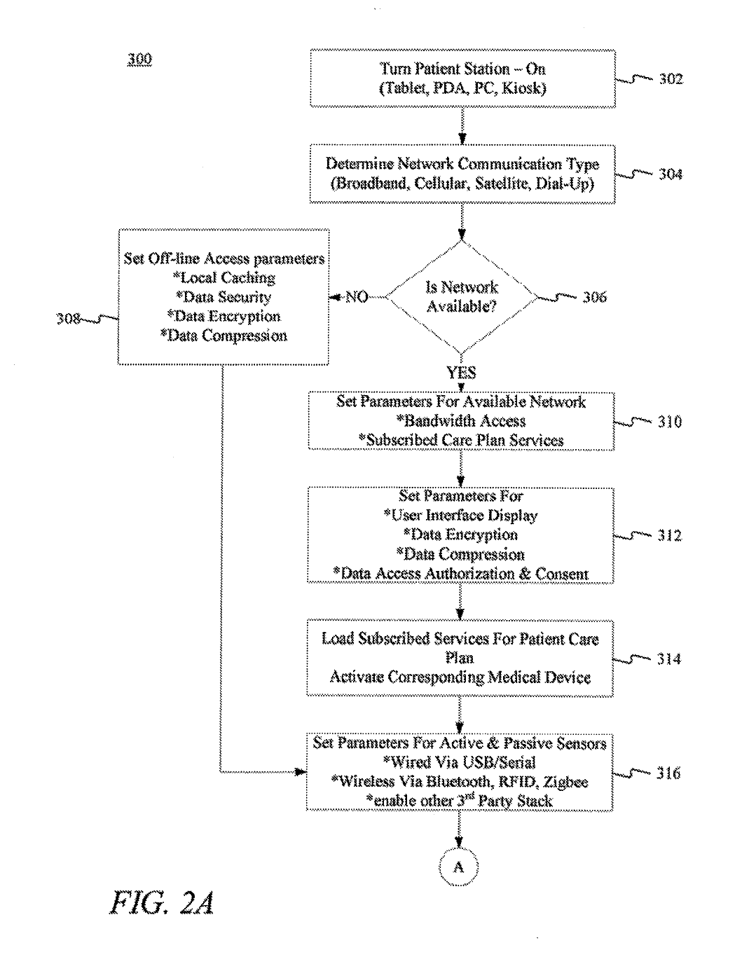

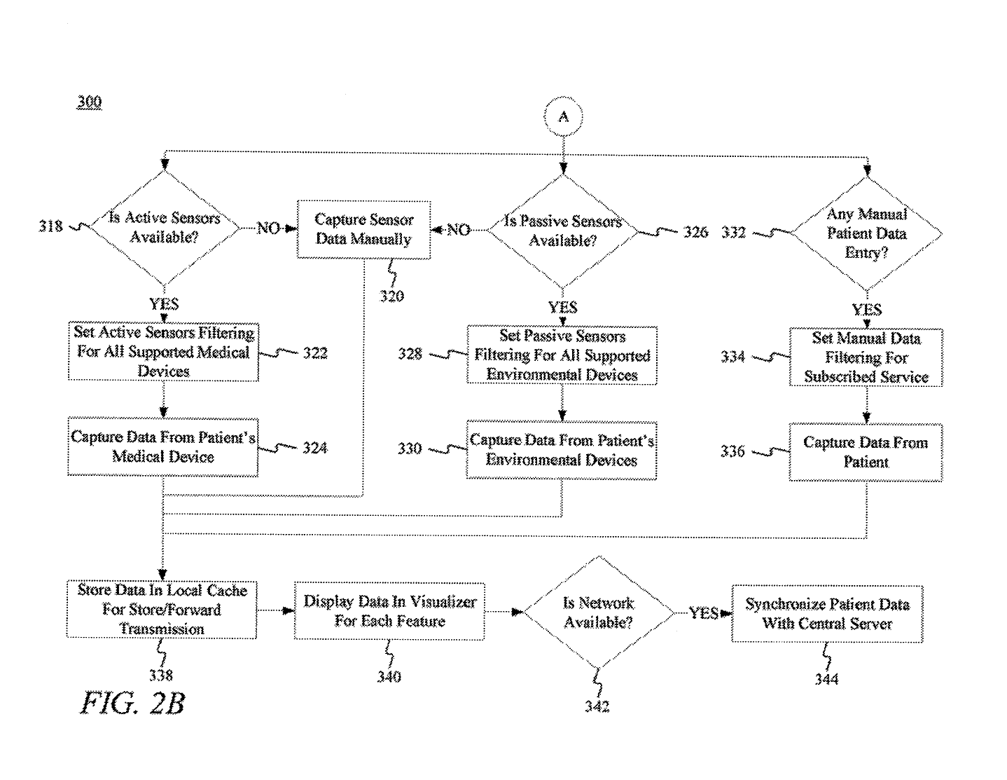

Referring now to FIG. 2A, the patient station which is a remote device utilized to enter patient physiological data remotely, may be any one of the following devices: a tablet PC, a PDA, a personal computer, a Kiosk, laptop or any other computer-implemented configuration including a display screen, processor and memory. When operating a patient station, initially the device must be turned on 302. Upon activating the patient station, a communications link test is performed 304 by a communications link module to determine the network communication type across which the patient station shall transmit patient data. It is to be understood that the network communication type may be a wide area network that includes dialup (56k), ISDN, T1, DSL, broadband, cellular, satellite, or any other communications medium that facilitates the transmission of data. The communications link module that checks the network communication type performs an assessment of which communication types may be available and also selects the optimal communications network if more than one communications network type is detected. For example, it is contemplated that there may be patient stations that include both dialup and broadband network communications. The communications link module that checks the network communication type selects the optimal network communication type and then determines whether the communications network selected is available 306. If the network is not available, the communications link module sets up the patient station to operate in offline mode 308.

During offline mode 308 the patient may still use the patient station, even though there is no network communication between the patient station and the remote healthcare server that functions as a central data repository for patient information. However, the patient may interact with the patient station graphical user interface application to input data manually and to facilitate automatic capture of data from active and passive sensors. Data input during offline mode is locally cached. Off line mode also facilitates setting of security on patient data, configuration of encryption and data compression technology being used. Alternatively, if the communications network is available 306, the patient station sets parameters for transmitting data across the available communications network. The parameters that shall be set are determined by the network communication type. Next, the patient station determines the type of care plan services the patient has access to 310. The care plan services may include services such as video visit, vital signs monitoring, blood pressure monitoring, blood glucose monitoring, blood oxygen monitoring, body weight monitoring, body temperature monitoring, pulmonary function analysis, respiratory monitoring, neurological monitoring, cardiac monitoring, sleep monitoring bathroom visit monitoring, bedroom visit monitoring, activity monitoring (sensors in the house), meal preparation monitoring air quality monitoring, patient fall status monitoring (sensors to detect body up/down position) or any other services that may be available to a patient via the patient workstation. It is to be understood that the care plan services that are active as icons on the patient station shall be configured by the care provider remotely or directly upon the patient station prior to delivery. The patient station is configured for the patient based on the patient's illnesses and the services that a patient may require. For example, if a patient is diabetic, the patient station shall be configured to interface with a glucose meter and a weight scale and have the medication reminder service. By way of further example, if the patient is a cardiac heart failure patient (CHF), the patient station may be configured to interface with a stethoscope as well as an apparatus for capturing the patient's ECG measurements.

Following a determination by the patient workstation that the network is available, a determination is made by the patient station configuration module of the bandwidth for the communications network and the services which may be pushed on that bandwidth 310. Next the system sets the patient station up for user interface display 312. If the network communication type is dialup, a patient would not be able to facilitate wound management interface, because wound management interface includes a video component. If the network communication is high-speed DSL, wound management is an application which may be engaged because the video component may be streamed via the high-speed DSL connection. For example a patient having diabetes, may subscribe to the wound management service and thereby have an active wound management icon display on the patient station. The wound management service allows wounds to be displayed and recorded by the healthcare provider. Typically during operation, a patient station camera is utilized to facilitate capture of ulcers on the feet of the patient for transmission back to the central server of the healthcare provider system. The images are transmitted from the patient station back to the central server of the healthcare provider system. A nurse stationed at a work station which is connected to the central server may view the images to provide feedback which may be immediate when images are viewed as they are being streamed across the communications network. The images may also be viewed at a later time when the video images are stored in server memory.

Next, the patient station configuration module sets the parameters for user interface display, data encryption, data compression, and data access, authorization and consent 312. The data encryption parameters being utilized is a key pair encryption. A key that is stored on the healthcare provider's server is utilized to encrypt the data. Utilization of key pair encryption guarantees that data transmitted over the communication network cannot be intercepted and viewed by individuals intercepting data being transmitted over the communications network. Data compression is performed to facilitate shrinking of data so that the data can be transmitted on a network having very low bandwidth. For example if the communications network is dial-up, the data may be compressed and transmitted at a faster rate. The compression algorithm is a standard application protocol interface (API). Data access, authorization and consent is the control mechanism whereby the system dictates the individuals who have access to and can actually look at the patient data once it is captured. The data access, authorization and consent parameters define the individuals whom may have access to patient data. Data access, authorization and consent parameters are defined by the patient through the patient station. For example a patient may define the parameters such that his or her pharmacist does not have access to the patient's physiological data representative of the patient's vital signs. However, the pharmacist may have access to data concerning a patient's diet, medication plan and any other data which the patient determines that the pharmacist needs to have access.

Next, services to which the patient subscribes are loaded onto the patient station by loading the icons that correspond to a subscribed service onto the patient station 314. Based on the icons loaded onto the patient station, active and passive sensors that correspond to the service icons loaded may be activated by engaging the icons. For example, an icon is loaded onto the patient workstation in order to facilitate glucose monitoring. That icon has to be operatively connected to a sensor, which in this example is an active sensor, such as a glucose monitor. For glucose monitoring interface to be fully functional on the patient station, the glucose monitor must be activated and operatively connected to the patient workstation. In one embodiment operative connection and activation may be performed by Bluetooth communications. Next, parameters are set for active and passive sensors 316. Engaging the subscriber service icon causes the parameters for the active and passive sensors to be set 316. It is contemplated that active and passive sensors may be connected or communicating with the patient station via wired USB or serial connections, wireless Bluetooth, RFID or Zigbee communications or any other third party communications protocol. The Bluetooth communications link is performed by pairing the workstation with the active or passive sensor in accordance with normal Bluetooth pairing protocol.

Following the setup of the parameters for active and passive sensors, in accordance with the services associated with a patient, the system tries to determine whether any active or passive sensors are available 318, 326. In the case of a diabetic patient they have engaged the icon for measuring their blood sugar level through use of the glucose monitor, an active sensor. Upon a determination that there are active sensors 318, a filtering mechanism 322 is engaged to make sure that only the proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also associated with data that has been flagged and the alert is transmitted to the remote central server and thereby to previously defined individuals to provide notice that something abnormal is occurring with the patient or the active sensors.

Upon a determination that there are passive sensors 326, a filtering mechanism 328 is engaged to make sure that only the proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also associated with the data that has been flagged and the alert is transmitted to the remote central server and thereby to previously defined individuals to provide notice that something abnormal is occurring with the patient or the sensors.

The system is also capable of facilitating manual data entry 332. For example if a patient needs to enter their temperature into the patient station, because thermometers are not Bluetooth capable nor do they have USB or any other communications capability, the user must enter data representative of the patient's temperature into the patient station manually. The patient station includes a keypad whereby the patient may enter the value that the patient sees on the medical device. Following a determination that there is data for manual data entry 332, a filtering mechanism 334 is engaged to make sure that only proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also attached to the data and the alert is transmitted to the remote healthcare provider system to indicate a potential patient health issue or a problem with the device for which data has been entered.

The patient data captured by the patient station is stored in a local cache for the store forward transmission function 338. The store forward function defines how much of a data stream needs to be stored in order to facilitate safe data transmission in order to allow for the recovery of data which may have been lost during a faulty transmission or service interruption. For example, the amount of data that needs to be stored in the local cache before being forwarded depends on whether data is to be transmitted across a broadband connection network or a dial up connection.

In one embodiment, when the communications network is dial up, data is stored in 10 second groupings and forwarded. When the communications network is broadband, data packets are stored in 30 second blocks and forwarded. The 30 second block of data packets are transmitted across the communications network in an orderly fashion. Patient station data processing includes an algorithm that tracks the data packets being sent and includes a verification mechanism for verifying that all data packets transmitted within a 30 second block were received. The verification mechanism is the transmission of an acknowledgement that is sent back to the patient station from the central server following verification by the algorithm that the entire 30 second block of data packets was received. The algorithm determines whether a block of data packets has been received by the size of the block of data packets. For example a first 30 second block of data packets is created then sent, a second 30 second block of data packets is created then sent, a third 30 second block of data packets is created then sent and so on. This helps facilitate maintaining the integrity of the data so that if there is a connection loss during transmission of the second 30 second block of data packets, no other data shall be transmitted until the connection has been reestablished. Upon reestablishing the connection, the entire second 30 second block of data packets shall be sent again and a third 30 second block of data packets will then be sent behind the second 30 second block of data packets in the previously defined sequence.

Next, the data for each service is displayed in a visualizer to facilitate graphic representation of captured patient data 340. Next the system checks to determine if the communications network is online or available 342. If the network is available the patient workstation synchronizes and transmits patient data with the central server 344.

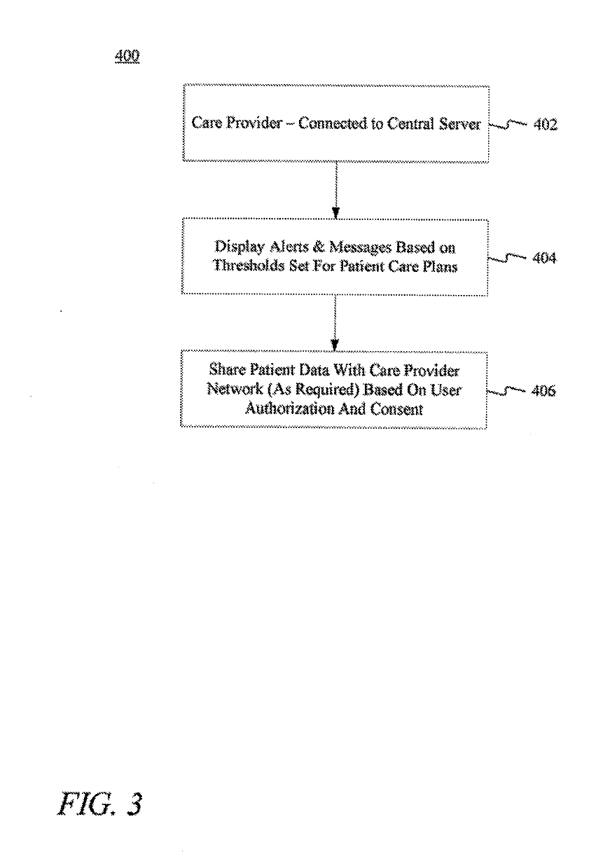

The central server 344 serves as a centralized data repository to which health care providers and other individuals who have been granted access authorization and consent by the patient to certain data files may connect and gain access to information to which they have authorization. As illustrated in FIG. 3, health care providers may connect to the central server 402. Connection may occur via WAN, but is generally done via a web based Internet connection. The application that manages connection to the host server is simply a web browser that individuals enter and gain access to in response to the entry of their respective credentials. Upon gaining access to the web browser, the user receives displays, alerts and messages based on their respective access authorization and consent previously defined by the patient 404. The web browser facilitates access to the centralized data repository by allowing users to login and gain access to files based on the authorization and consent provided a user by the patient 406. The health care provider seeking access to the central server may be a network of care providers including any of the following individuals: nurse, primary physician, pharmacist, family members, etc. These individuals each have access to certain subsets of the patient data based on the authority assigned at the access authorization and consent previously defined 406.

While certain features and embodiments of the invention have been described, other embodiments of the invention will be apparent to those skilled in the art from consideration of the specification and practice of the embodiments of the invention disclosed herein. Furthermore, although embodiments of the present invention have been described as being associated with data stored in memory and other storage mediums, one skilled in the art will appreciate that these aspects can also be stored on or read from other types of computer-readable media, such as secondary storage devices, like hard disks, floppy disks, or a CD-ROM, a carrier wave from the Internet, or other forms of RAM or ROM. Further, the steps of the disclosed methods may be modified in any manner, including by reordering steps and/or inserting or deleting steps, without departing from the principles of the invention.

It is intended, therefore, that the specification and examples be considered as exemplary only, with a true scope and spirit of the invention being indicated by the following claims and their full scope of equivalents.

What is claimed is:

1. A patient health system comprising:
a patient data collection system; and
a provider analysis system,
wherein the patient health system is configured for transmission of patient health data from the patient data collection system to the provider analysis system,
a. the patient data collection system, located proximate a patient, configured to collect patient physiological data, and operatively coupled to a communications network;
b. the provider analysis system coupled to the communications network and located remote from the patient data collection system;
wherein the patient data collection system, located proximate a patient, is comprised an environmental data collection device couple to a patient computing station, the patient computing station including a configuration module for:
identifying at least one communication links between the patient computing station and the provider analysis system;
assessing the bandwidth of each communication link identified; and
selecting an optimal communications link based on the type of data to be transmitted to the provider analysis system, wherein the communications link selected has a bandwidth sufficient to handle transmission of data associated with a medical procedure administered, wherein the computing station is configured to determine the reliability of the communications network and transmit patient physiological data upon a determination that the communications network is reliable.

2. A patient health system comprising:
a patient data collection system; and
a health provider analysis system;
wherein the patient health system provides for transmission of patient heart beat data collected and stored within data storage of the patient data collection system to the health provider analysis system, wherein
the patient collection system, located proximate a patient, is configured to collect patient physiological data representative of a patient's heart beat, and operatively coupled to a communications network;
wherein the health care provider analysis system is coupled to the communications network and located remote from the patient heart beat data collection system;
wherein the patient data collection system is comprised of an electronic stethoscope coupled to a patient work station, wherein the patient work station is configured to determine the reliability of the communications network and transmit patient physiological data upon a determination that the communications network is reliable.

3. The patient workstation of claim 2 comprising a data input device, data display device, processor, memory and an audio transceiver.

4. The patient workstation of claim 2 whereby the memory includes a temporary memory buffer.

5. The patient workstation of claim 2 configured to provide the patient with instructions regarding positioning of stethoscope audio receiver on the patient.

6. A computer implemented method of transmitting data representative of a patient's heartbeat from a patient data collection system located proximate a patient to a health care provider analysis system comprising:
a. providing a sensor proximate the patient to sense the heart beat of a patient and generate the data representative of a patient's heartbeat;
b. the patient data collection system processes the data representative of a patient's heartbeat to determine if it is within an acceptable range;
c. the patient data collection system identifies by flagging portions of the data representative of a patient's heartbeat that is outside of the acceptable range;
d. the patient data collection system stores the data representative of a patient's heartbeat along with any flagged portions in a memory buffer within the patient data collection system;
e. the patient data collection system determines if a communications medium between the patient data collections system and the health care provider analysis system is sufficiently stable for data transmission and transmits the data representative of a patient's heartbeat along with any flagged portions to the health care provider analysis system upon a determination that the communications medium is stable.

7. The method of claim 6 wherein the sensor is a stethoscope.

8. The method of claim 6 wherein a first audio transceiver is further provided proximate the patient to facilitate audio communications between the patient and the health care provider analysis system that includes an audio transceiver.

9. The method of claim 6 wherein the data transmitted to the healthcare provider analysis system over a communications medium is digital data.

10. The method of claim 6 wherein the communications medium comprises a wireless network.

11. The method of claim 6 wherein the communications medium comprises a broadband network.
